(19) United States
(12) Patent Application Publication
(10) Pub. No.: US 2015/0265859 A1
(43) Pub. Date: Sep. 24, 2015

(54) OXYGEN BELT BREATHING PACK

(71) Applicant: Stan A. Sanders, Chesterfield, MO (US)

(72) Inventor: Stan A. Sanders, Chesterfield, MO (US)

(21) Appl. No.: 14/552,077

(22) Filed: Nov. 24, 2014

Related U.S. Application Data
(60) Provisional application No. 61/908,350, filed on Nov. 25, 2013.

(51) Int. Cl. A62B 25/00; A62B 9/04; A62B 7/02; A61M 16/08; A61M 16/20

(52) U.S. Cl. CPC A62B 25/00; A61M 16/0875; A61M 16/20; A61M 16/0816; A62B 7/02; A62B 9/04; A61M 2209/088

(57) ABSTRACT

An oxygen belt breathing pack includes at least one flexible polymeric pressure vessel connected with sections of flexible conduit to either a sealing fitting, an inlet fitting, another section of flexible conduit attached to another pressure vessel or a manifold. The manifold provides connections for a high pressure regulator and oxygen fill source. The pressure vessel and sections of flexible conduit are encased in high strength fiber material. The pressure vessel is wrapped in high strength ballistic ribbon material. The high pressure regulator is connected to a low pressure hose connected to a nasal cannula. A flexible container is formed of resilient material, and is sized and shaped to accommodate the at least one pressure vessel, the sections of flexible conduit, the manifold and to provide storage for the nasal cannula. The container is formed as a belt or lightweight backpack. A pressure gauge monitors oxygen use by the patient.

### FIELD OF INVENTION

This invention relates to the field of portable oxygen breathing equipment, and more specifically to wearable breathing systems comprised of light weight synthetic pressure vessels and related components.

### BACKGROUND OF THE INVENTION

Patients that require supplementary oxygen on a routine basis are amongst those least able to cope with the weight and bulk of present day portable oxygen systems. Typically these systems consist of a heavy metallic reservoir mounted to a wheeled trolley or back pack. The system also includes a fill valve, pressure regulator, delivery hose and cannula for provision of oxygen to the patient's nose. These systems typically weigh 6-9 pounds, operate at approximately 2000 psi and can provide up to three hours breathing time at a typical delivery rate. The weight and bulk of these systems make them awkward to transport and use. In addition, a certain stigma attaches to the use of these bulky systems and tends to discourage those who need them from being out in public.

Various designs have been attempted to address some of these problems:

U.S. Pat. No. 7,156,094, issued to Chornyj, is directed to a breathing apparatus and pressure vessels therefore. The garment is a wearable garment with a plurality of compartments disposed about the garment to receive air storage vessels along with the necessary regulators and connecting hoses so that the user may receive an air supply from the storage vessels. The vest apparatus comprises a series of high pressure vessels that are interconnected and contained within a bag. Pressure monitoring sensors and alarms are also included. The pressure vessels are made of a non-metal material in order to reduce the weight and may be capable of sustaining pressures in the range of 4500-7500 PSI. The non-metal material is a carbon fiber epoxy material with a rubber or nylon coating on the inner surface and a second rubber or nylon coating on the outer surface thereof. The shape of the vessels may be a traditional cylindrical shape, but in this case they are made more elliptical so as to more closely fit the user.

U.S. Pat. No. 6,513,522, issued to Izuchukwu et al. is directed to a wearable storage system for pressurized fluids and may be used as part of scuba diving equipment and other portable, pressurized oxygen supplies for various applications. In two embodiments of interest are seen wherein a vest includes front panel portions along with a back panel portion that includes a pressure vessel and interconnected chambers. Appropriate valves and regulators are also described.

U.S. Pat. No. 5,127,399, issued to Scholley is directed to flexible containers for compressed gases which may be carried on a person for various applications including diving. Conventional containers for this purpose are normally cylindrical in shape and usually made of steel or aluminum and thus are cumbersome to wear and relatively heavy in weight. The containers described in this Patent are flexible and adaptable to be worn over or with over garments, and thus more convenient.

U.S. Pat. No. 3,428,961, issued to Schueller is directed an emergency respiration apparatus and is simply a lightweight self-contained unit that supplies pressurized breathable gas to be incorporated into a vest or other garment for use in hazardous work areas.

U.S. Pat. No. 65,760, issued to McKeen is directed a diving apparatus and again shows a suit-like garment that may be pressurized and utilized in diving operations. The important embodiments are seen in FIGS. 2 and 3 wherein the pressurized portions are in the form of tubular air-chambers E. Thus, these suits may be worn by a diver keeping hands and arms free to do the necessary work during the diving session.

It is an objective of the present invention to provide a lightweight, compact, oxygen supply and associated breathing apparatus for the ambulatory care patient. It is a further objective to provide such a system that allows the apparatus to be as unobtrusive as possible. It is a still further objective of the invention to use polymeric pressure vessels that will not explode when exposed to heat, cold or crushing force. It is yet a further objective to provide pressure vessels that will dissipate pressure in a controlled manner. It is another objective of the present invention to provide a breathing apparatus that can be worn comfortably for long periods of time. Finally, it is an objective to provide a system that can be rapidly filled, is durable, and is inexpensive to produce.

While some of the objectives of the present invention are disclosed in the prior art, none of the inventions found include all of the requirements identified.

### SUMMARY OF THE INVENTION

The present invention addresses all of the deficiencies of prior art oxygen belt breathing pack inventions and satisfies all of the objectives described above.

(1) An oxygen belt breathing pack providing the desired features may be constructed from the following components. At least one flexible polymeric pressure vessel is provided. The vessel has a first end and a second end. Each of the first and second ends has an attached section of flexible conduit. Each of the sections of flexible conduit is attached to either of a sealing fitting, an inlet fitting, another section of flexible conduit attached to another vessel or a manifold. The at least one pressure vessel and the sections of flexible conduit are encased in high strength fiber material. The at least one pressure vessel is wrapped with a high strength ballistic ribbon material. A manifold is provided. The manifold is connected to the section of flexible conduit connected to the at least one pressure vessel. The manifold provides connections for a high pressure regulator and an oxygen fill source. A high pressure regulator is provided. A low pressure hose is connected to the high pressure regulator and a nasal cannula is connected to the low pressure hose. A flexible container is provided. The container is formed of resilient material, and is sized and shaped to accommodate the at least one pressure vessel, the sections of flexible conduit and the manifold.

(2) In a variant of the invention, the at least one pressure vessel includes any of an elongated cylindrical shape, an ovoid shape and a spherical shape.

(3) In another variant, the at least one pressure vessel includes a tube coiled in a compact shape.

(4) In still another variant, the high strength fiber material is selected from the group consisting of: rayon, nylon, glass or Kevlar® (aramid) fiber.

(5) In yet another variant, the high strength ballistic ribbon material is selected from the group consisting of prepreg carbon fiber or prepreg glass fiber.

(6) In a further variant, the high pressure regulator is an integral portion of the manifold.

(7) In still a further variant, the manifold further includes an overpressure rupture fitting.

(8) In yet a further variant, the at least one pressure vessel includes an overpressure rupture fitting.

(9) In another variant of the invention, a pressure transducer is provided. The pressure transducer detects pressure levels within the at least one pressure vessel and includes a wireless signal transmission mechanism.

(10) In still another variant, a remotely mounted pressure display device is provided. The display device receives wireless signals from the pressure transducer and displays a pressure reading.

(11) In yet another variant, the at least one polymeric pressure vessel is formed as a seamless cylindrical body having semi-spherical ends. Each of the ends has a central opening connected to the sections of flexible conduit.

(12) In a further variant, the at least one polymeric pressure vessel is formed as a pair of two part shells, the shells are welded together and reinforced with a two part ring assembly.

(13) In still a further variant, the at least one polymeric pressure vessel is formed as an elongated tube with reduced diameter ends for connection to either of coupling, inlet and sealing fittings.

(14) In yet a further variant, the at least one polymeric pressure vessel further includes at least one intermediate section. The intermediate section has a reduced diameter to provide increased flexibility when forming the pressure vessel into a compact shape to fit a container.

(15) In another variant of the invention, the at least one polymeric pressure vessel is formed as an elongated, corrugated tube with reduced diameter ends for connection to either of coupling, inlet and sealing fittings.

(16) In still another variant, the at least one polymeric pressure vessel further includes at least one intermediate section. The intermediate section has a reduced diameter to provide increased flexibility when forming the pressure vessel into a compact shape to fit a container.

(17) In yet another variant, a concentrator is provided. The concentrator includes a compressor. The compressor has a rotational power source, a low pressure input and a high pressure output. At least one concentrator polymeric pressure vessel is provided. The pressure vessel is fluidly connected to the high pressure output. A high pressure regulator is provided. The regulator is fluidly connected to the pressure vessel. A high pressure connection is provided. The high pressure connection is fluidly connected to the high pressure regulator and connectable to an external pressure vessel.

(18) In a further variant, the concentrator further includes an inlet oxygen filter and a pressure gauge. The pressure gauge measures pressure in the at least one concentrator pressure vessel. The concentrator includes a shut-off valve. The shut-off valve controls the high pressure connection.

(19) In still a further variant, the at least one concentrator pressure vessel is formed as a seamless cylindrical body having semi-spherical ends. Each of the ends has a central opening connected to the sections of flexible conduit.

(20) In yet a further variant, the at least one concentrator pressure vessel is formed as a pair of two part shells. The shells are welded together and reinforced with a two part ring assembly.

(21) In another variant of the invention, the at least one concentrator pressure vessel is formed as an elongated tube with reduced diameter ends for connection to either of coupling, inlet and sealing fittings.

(22) In still another variant, the at least one concentrator pressure vessel further includes at least one intermediate section. The intermediate section has a reduced diameter to provide increased flexibility when forming the pressure vessel into a compact shape to fit a container.

(23) In yet another variant, the at least one concentrator pressure vessel is formed as an elongated, corrugated tube with reduced diameter ends for connection to either of coupling, inlet and sealing fittings.

(24) In a further variant, the at least one concentrator pressure vessel further includes at least one intermediate section. The intermediate section has a reduced diameter to provide increased flexibility when forming the pressure vessel into a compact shape to fit a container.

(25) In still a further variant, the flexible container is in the form of a belt that can be worn about the waist of an ambulatory care patient.

(26) In yet a further variant, the high pressure regulator includes a demand valve to supply oxygen directly to said nasal cannula at a reduced pressure.

(27) In a final variant, a pressure gauge is provided. The gauge is mounted to the manifold.

An appreciation of the other aims and objectives of the present invention and an understanding of it may be achieved by referring to the accompanying drawings and the detailed description of a preferred embodiment.

### DESCRIPTION OF THE DRAWINGS

FIG. 1 is a perspective view of polymetric pressure vessels, manifold and pressure regulator of the preferred embodiment of the invention;

FIG. 2 is a side elevational view of the FIG. 1 embodiment within an outline representing the oxygelt belt breathing pack;

FIG. 3 is a side elevational view of a second embodiment of polymetric pressure vessels within an outline representing the oxygen belt breathing pack;

FIG. 3A is a partial close-up side elevational view of the pressure vessel of the FIG. 3 embodiment;

FIG. 3B is a partial close-up side elevational view of a corrugated pressure vessel for use with the FIG. 3 embodiment;

FIG. 4 is a side elevational view of a plurality of linked polymetric pressure vessels for use with the oxygen belt breathing pack;

FIG. 5 is a partial close-up side elevational view of a corrugated pressure vessel for use with the FIG. 1 embodiment illustrating winding of the pressure vessel and flexible conduit with high strength fiber material;

FIG. 6 is a partial close-up side elevational view of a corrugated pressure vessel for use with the FIG. 1 embodiment illustrating winding of the pressure vessel and flexible conduit with high strength fiber material and winding of the pressure vessel with a high strength ballistic ribbon material;

FIG. 7 is rear elevational view of the oxygen belt breathing pack and nasal cannula;

FIG. 8 is rear elevational view of the oxygen belt breathing pack and nasal cannula as worn by a patient;

FIG. 9 is a close-up perspective view of the manifold, oxygen filling fitting and pressure regulator;

FIG. 10 is a partial sectional side elevational view of another embodiment of the polymeric pressure vessel illustrating an ovoid shape and an overpressure rupture fitting;

FIG. 11 is a top view of a plurality of round polymetric pressure vessels;

FIG. 12 partial cross-sectional side elevational view of the FIG. 11 pressure vessels taken along the line 12-12;

FIG. 13 is a side elevational view of an alternative embodiment of a pressure vessel formed as a seamless cylindrical body that has semi-spherical ends;

FIG. 14 is an exploded side elevational view of an alternative embodiment of a pressure vessel formed as a pair of two part shells;

FIG. 15 is a perspective view of the FIG. 14 embodiment welded together and reinforced with a two part ring assembly;

FIG. 16 is a side elevational view of a corrugated tubular polymeric flexible pressure vessel;

FIG. 17 is a side elevational view of a pair of corrugated tubular polymeric flexible pressure vessels connected by a flexible conduit;

FIG. 18 is an enlarged detail of one end of the FIG. 16 embodiment;

FIG. 19 is a side elevational view of a concentrator for use with the oxygen belt breathing pack; and

FIG. 20 is a side elevational view of a plurality of coiled, cylindrical polymeric flexible pressure vessels for use with the concentrator.

### DETAILED DESCRIPTION OF THE PREFERRED EMBODIMENT

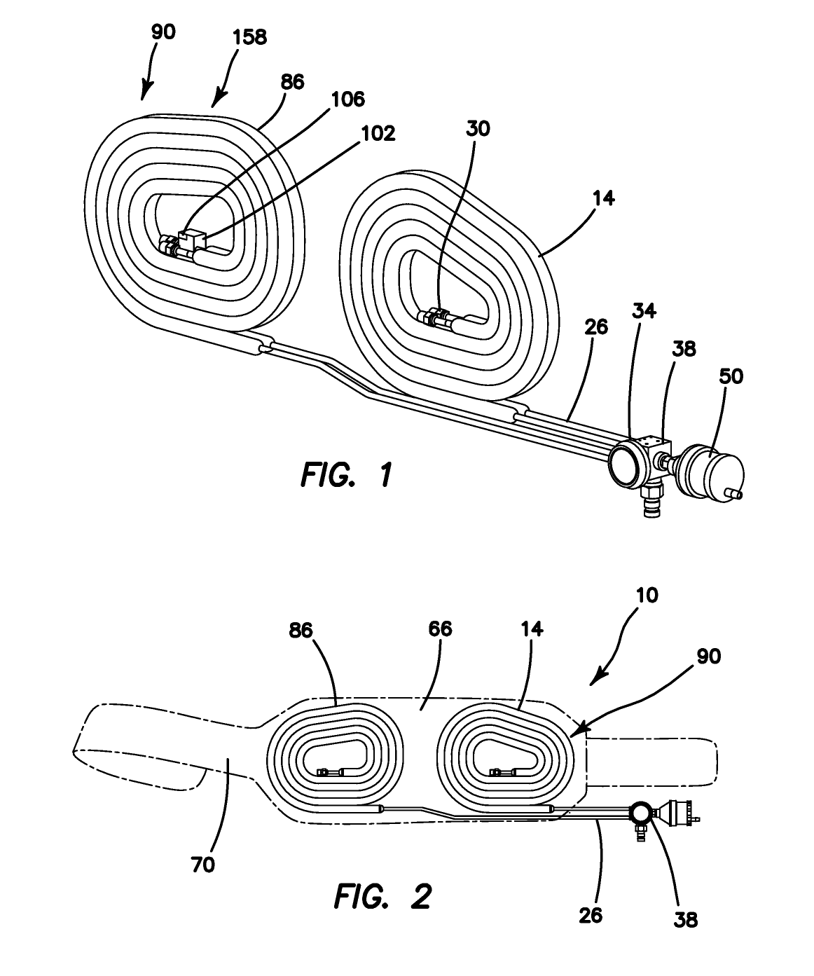

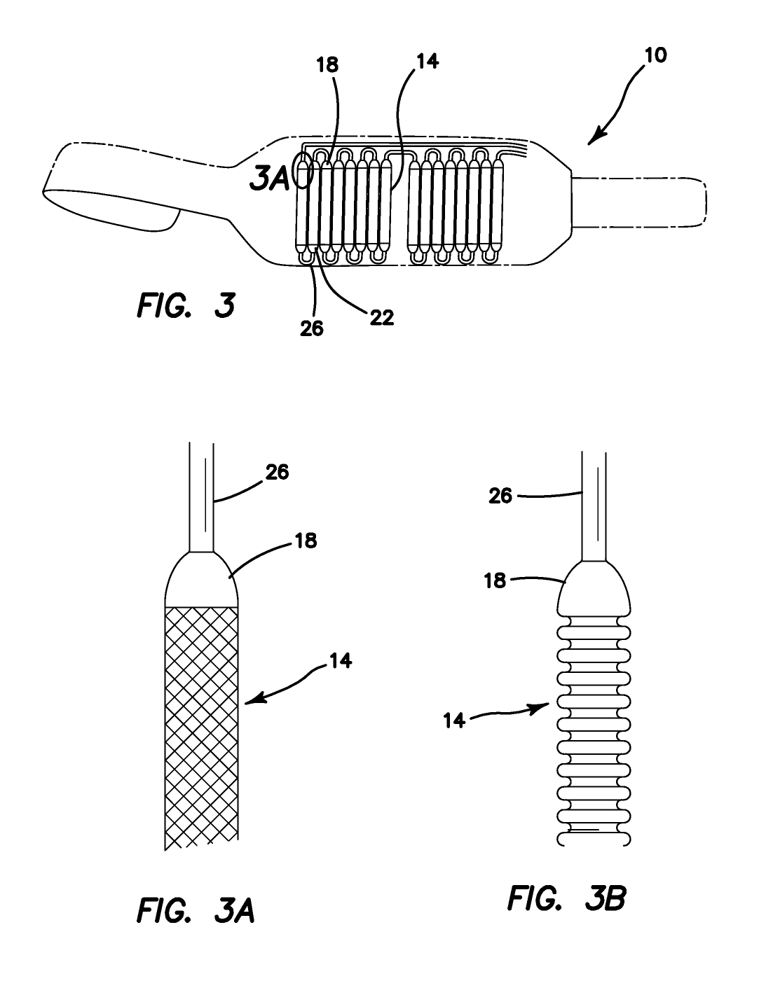

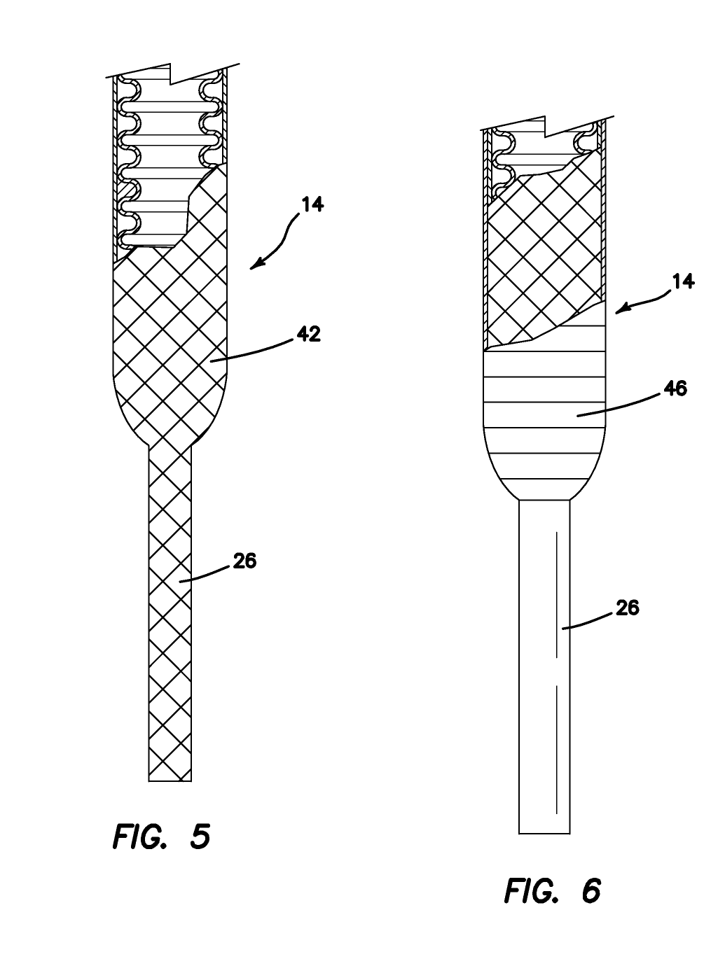

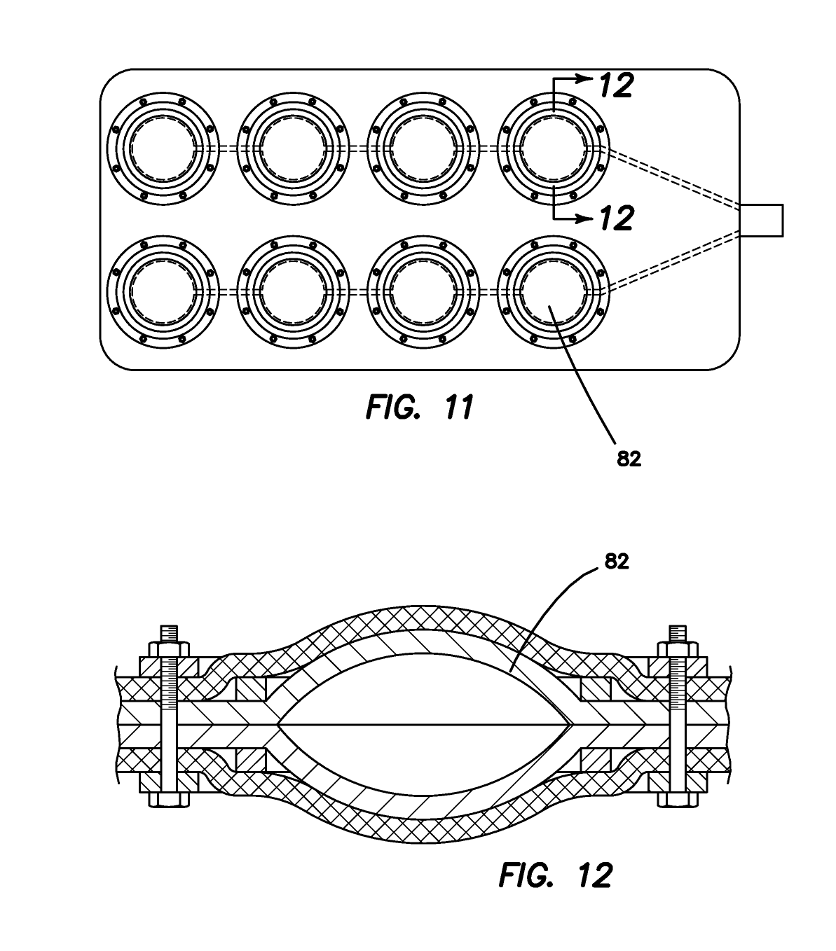

(1) FIGS. 1-20 illustrate an oxygen belt breathing pack 10 providing the desired features that may be constructed from the following components. As illustrated in FIGS. 1-4, at least one flexible polymeric pressure vessel 14 is provided. The vessel has a first end 18 and a second end 22. Each of the first 18 and second 22 ends has an attached section of flexible conduit 26. Each of the sections of flexible conduit 26 is attached to either of a vessel 14 or a manifold 38. As illustrated in FIGS. 5 and 6, the at least one pressure vessel 14 and the sections of flexible conduit 26 are encased in high strength fiber material 42. The at least one pressure vessel 14 is wrapped with a high strength ballistic ribbon material 46. As illustrated in FIGS. 1-3 and 7-9, a manifold 38 is provided. The manifold 38 is connected to the section of flexible conduit 26 connected to the at least one pressure vessel 14. The manifold 38 provides connections for a high pressure regulator 50 and an oxygen fill source 54. A high pressure regulator 50 is provided. A low pressure hose 58 is connected to the high pressure regulator 50 and a nasal cannula 62 is connected to the low pressure hose 58. A flexible container 66 is provided. The container 66 is formed of resilient material 70, and is sized and shaped to accommodate the at least one pressure vessel 14, the sections of flexible conduit 26 and the manifold 38.

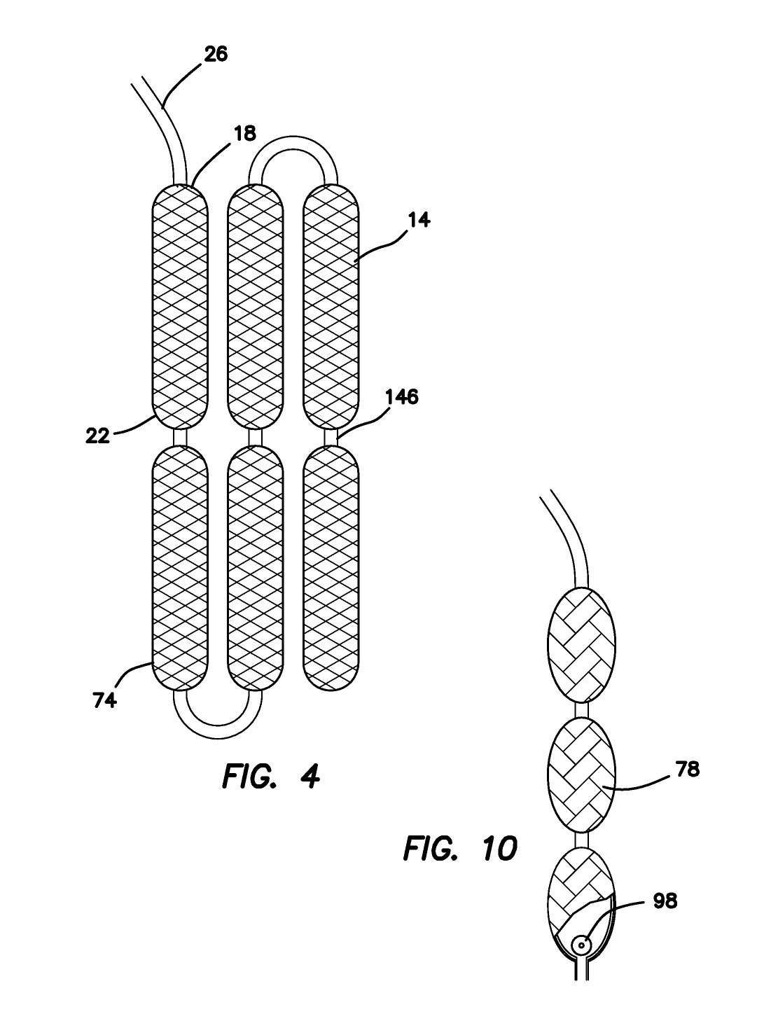

(2) In a variant of the invention, as illustrated in FIGS. 4 and 10-12, the at least one pressure vessel 14 includes any of an elongated cylindrical shape 74, an ovoid shape 78 and a spherical shape 82.

(3) In another variant, as illustrated in FIGS. 1 and 2, the at least one pressure vessel 14 includes a tube 86 coiled in a compact shape 90.

(4) In still another variant, the high strength fiber material 42 is selected from the group consisting of: rayon, nylon, glass or Kevlar® (aramid) fiber.

(5) In yet another variant, the high strength ballistic ribbon material 46 is selected from the group consisting of prepreg carbon fiber or prepreg glass fiber.

(6) In a further variant, the high pressure regulator 50 is an integral portion of the manifold 38 (not shown).

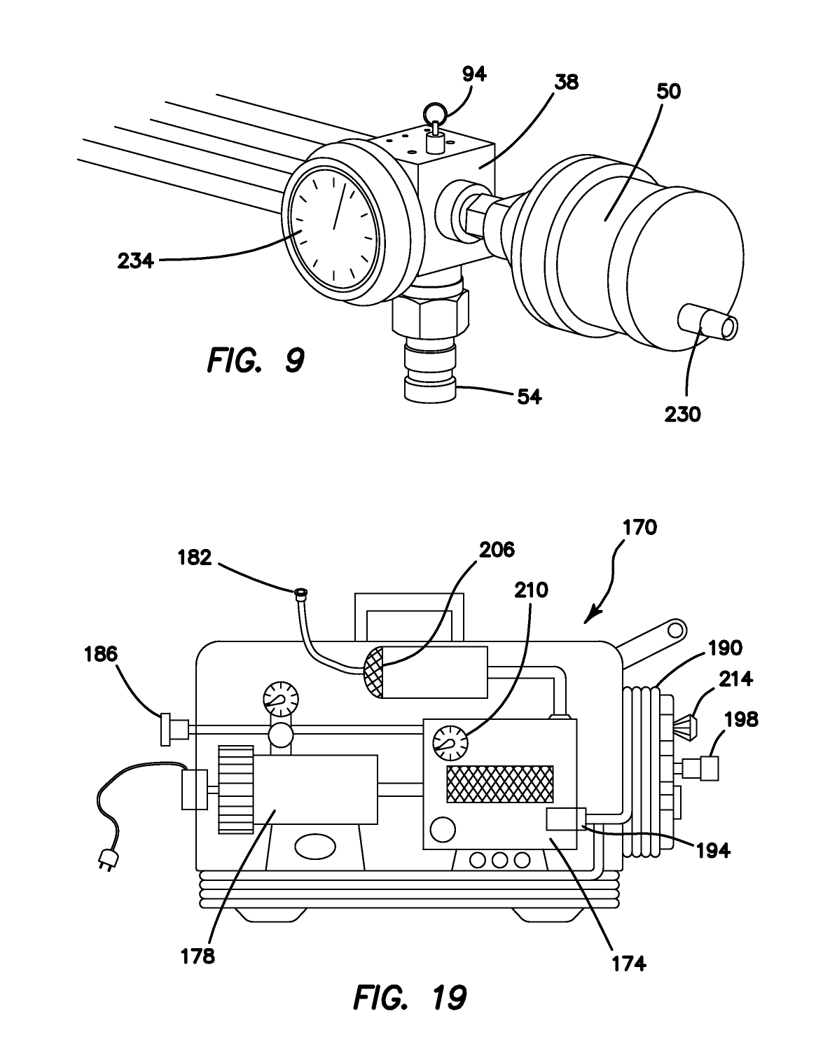

(7) In still a further variant, as illustrated in FIG. 9, the manifold 38 further includes an overpressure rupture fitting 94.

(8) In yet a further variant, as illustrated in FIG. 10, the at least one pressure vessel 14 includes an overpressure rupture fitting 98.

(9) In another variant of the invention, as illustrated in FIG. 1, a pressure transducer 102 is provided. The pressure transducer 102 detects pressure levels within the at least one pressure vessel 14 and includes a wireless signal transmission mechanism 106.

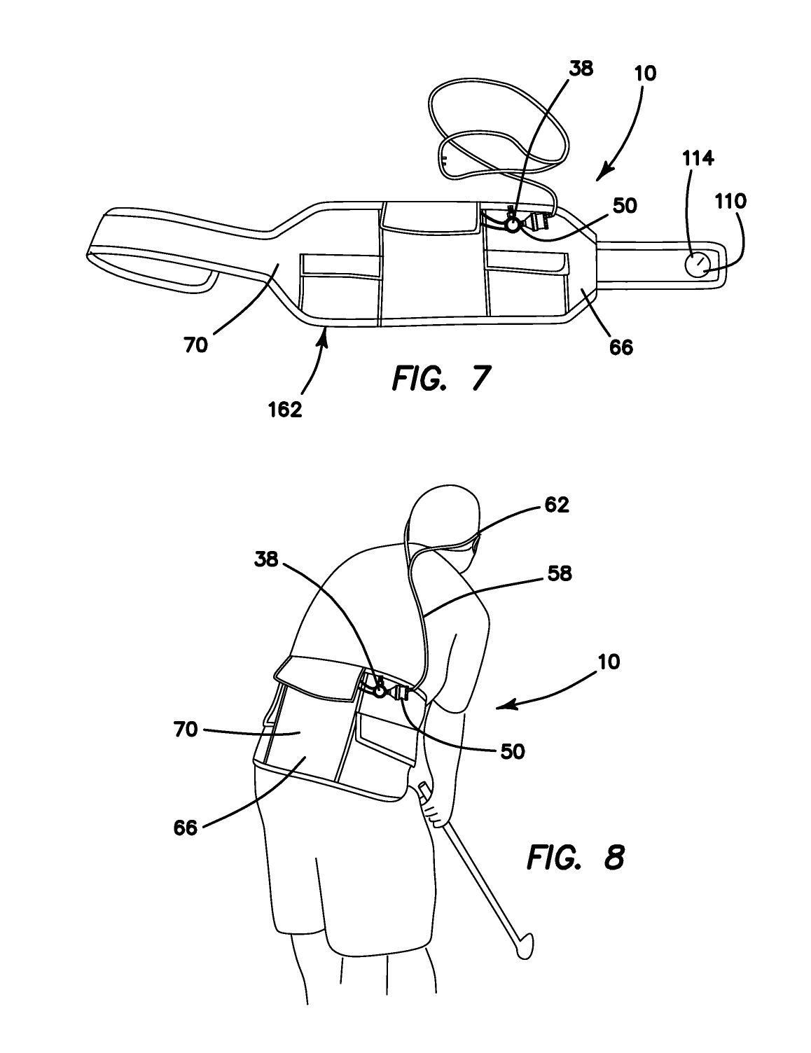

(10) In still another variant, as illustrated in FIG. 7, a remotely mounted pressure display device 110 is provided. The display device 110 receives wireless signals from the pressure transducer 102 and displays a pressure reading 114.

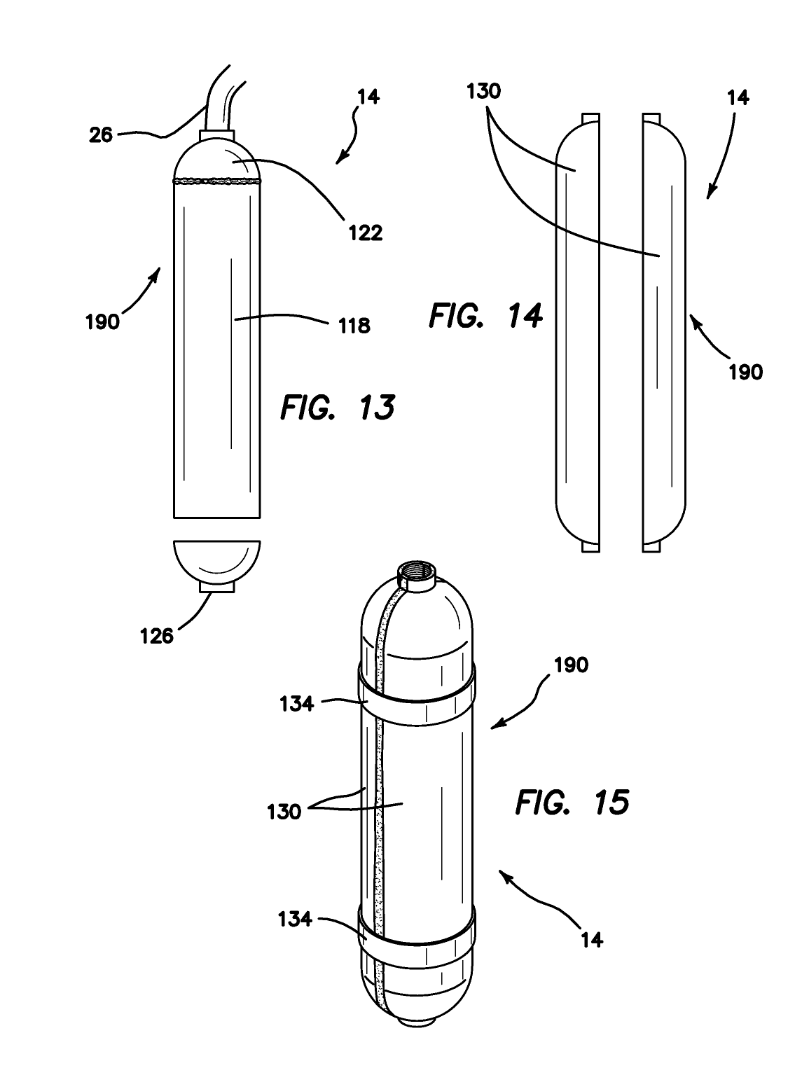

(11) In yet another variant, as illustrated in FIG. 13, the at least one polymeric pressure vessel 14 is formed as a seamless cylindrical body 118 having semi-spherical ends 122. Each of the ends 122 has a central opening 126 connected to the sections of flexible conduit 26.

(12) In a further variant, as illustrated in FIGS. 14 and 15, the at least one polymeric pressure vessel 14 is formed as a pair of two part shells 130, the shells 130 are welded together and reinforced with a two part ring assembly 134.

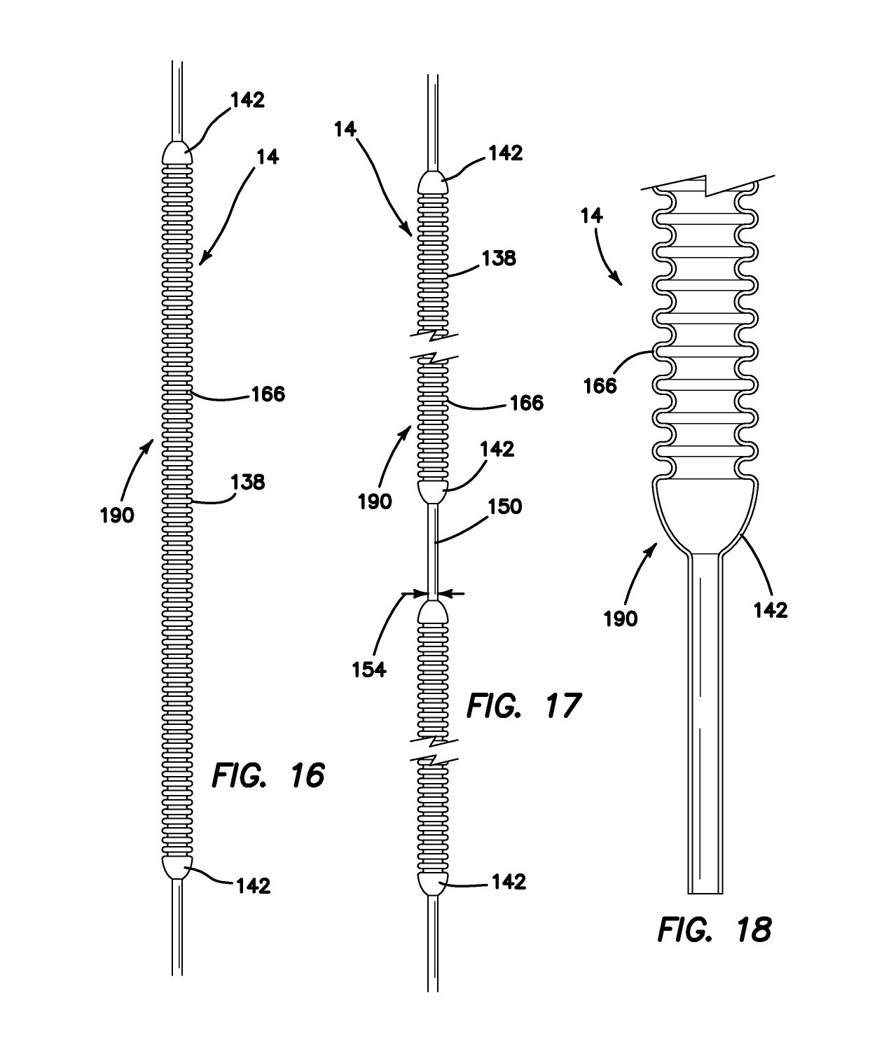

(13) In still a further variant, as illustrated in FIG. 16-18, the at least one polymeric pressure vessel 14 is formed as an elongated tube 138 with reduced diameter ends 142 for connection to either of coupling 146, inlet 34 and sealing fittings 30.

(14) In yet a further variant, as illustrated in FIG. 17, the at least one polymeric pressure vessel 138 further includes at least one intermediate section 150. The intermediate section 150 has a reduced diameter 154 to provide increased flexibility when forming the pressure vessel 138 into a compact shape 158 to fit a container 162.

(15) In another variant of the invention, as illustrated in FIG. 16-18, the at least one polymeric pressure vessel 14 is formed as an elongated, corrugated tube 166 with reduced diameter ends 142 for connection to either of coupling 146, inlet 34 and sealing fittings 30.

(16) In still another variant, as illustrated in FIG. 17, the at least one polymeric pressure vessel 166 further includes at least one intermediate section 150. The intermediate section 150 has a reduced diameter 154 to provide increased flexibility when forming the pressure vessel 138 into a compact shape 158 to fit a container 162.

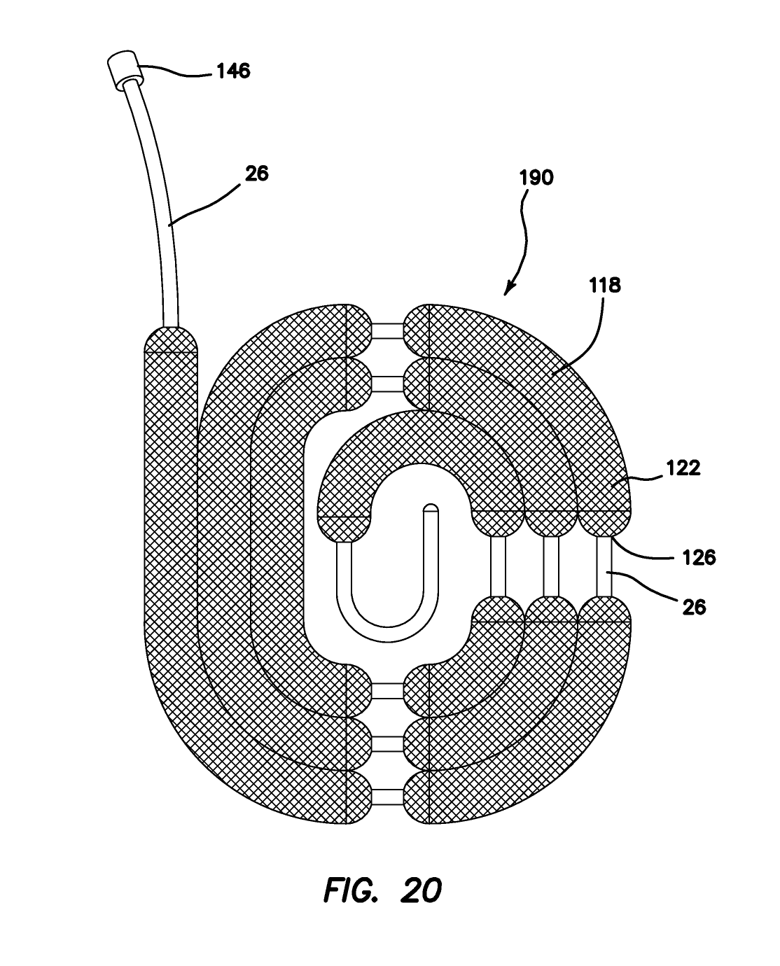

(17) In yet another variant, as illustrated in FIGS. 19 and 20, a concentrator 170 is provided. The concentrator 170 includes a compressor 174. The compressor 174 has a rotational power source 178, a low pressure input 182 and a high pressure output 186. At least one concentrator polymeric pressure vessel 190 is provided. The pressure vessel 190 is fluidly connected to the high pressure output 186. A high pressure regulator 194 is provided. The regulator 194 is fluidly connected to the pressure vessel 190. A high pressure connection 198 is provided. The high pressure connection 198 is fluidly connected to the high pressure regulator 194 and connectable to an external pressure vessel 14.

(18) In a further variant, the concentrator 170 further includes an inlet oxygen filter 206 and a pressure gauge 210. The pressure gauge 210 measures pressure in the at least one concentrator pressure vessel 190. The concentrator 170 includes a shut-off valve 214. The shut-off valve 214 controls the high pressure connection 198.

(19) In still a further variant, as illustrated in FIGS. 13 and 20, the at least one concentrator pressure vessel 190 is formed as a seamless cylindrical body 118 having semi-spherical ends 122. Each of the ends 122 has a central opening 126 connected to the sections of flexible conduit 26.

(20) In yet a further variant, as illustrated in FIGS. 14 and 15, the at least one concentrator pressure vessel 190 is formed as a pair of two part shells 130, the shells 130 are welded together and reinforced with a two part ring assembly 134.

(21) In another variant of the invention, as illustrated in FIGS. 16-18, the at least one concentrator pressure vessel 190 is formed as an elongated tube 138 with reduced diameter ends 142 for connection to either of coupling 146, inlet 34 and sealing fittings 30.

(22) In still another variant, as illustrated in FIG. 17, the at least one concentrator pressure vessel 190 further includes at least one intermediate section 150. The intermediate section 150 has a reduced diameter 154 to provide increased flexibility when forming the pressure vessel 138 into a compact shape 158 to fit a container 162.

(23) In yet another variant, as illustrated in FIGS. 16-18, the at least one concentrator pressure vessel 190 is formed as an elongated, corrugated tube 166 with reduced diameter ends 142 for connection to either of coupling 146, inlet 34 and sealing fittings 30.

(24) In a further variant, as illustrated in FIG. 17, the at least one concentrator pressure vessel 190 further includes at least one intermediate section 150. The intermediate section 150 has a reduced diameter 154 to provide increased flexibility when forming the pressure vessel 138 into a compact shape 158 to fit a container 162.

(25) In still a further variant, as illustrated in FIGS. 2, 7 and 8, the flexible container 66 is in the form of a belt 218 that can be worn about the waist 222 of an ambulatory care patient 226.

(26) In yet a further variant, as illustrated in FIG. 9, the high pressure regulator 50 includes a demand valve 230 to supply oxygen directly to said nasal cannula 62 at a reduced pressure.

(27) In a final variant, a pressure gauge 234 is provided. The gauge 234 is mounted to the manifold 38.

The oxygen belt breathing pack 10 has been described with reference to particular embodiments. Other modifications and enhancements can be made without departing from the spirit and scope of the claims that follow.

1. An oxygen belt breathing pack, comprising:
at least one flexible polymeric pressure vessel, said vessel having a first end and a second end;
each of said first and second ends having an attached section of flexible conduit;
each of said sections of flexible conduit being attached to either of a sealing fitting, an inlet fitting, another section of flexible conduit attached to another vessel and a manifold;
said at least one pressure vessel and said sections of flexible conduit being encased in high strength fiber material;
said at least one pressure vessel being wrapped with a high strength ballistic ribbon material;
a manifold, said manifold connected to said section of flexible conduit connected to said at least one pressure vessel, and providing connections for a high pressure regulator and an oxygen fill source;
a high pressure regulator, a low pressure hose connected to said high pressure regulator and a nasal cannula connected to said low pressure hose;
a flexible container, said container being formed of resilient material, and being sized and shaped to accommodate said at least one pressure vessel, said sections of flexible conduit and said manifold.

2. The oxygen belt breathing pack, as described in claim 1, wherein said at least one pressure vessel comprises any of an elongated cylindrical shape, an ovoid shape and a spherical shape.

3. The oxygen belt breathing pack, as described in claim 1, wherein said at least one pressure vessel comprises a tube coiled in a compact shape.

4. The oxygen belt breathing pack, as described in claim 1, wherein said high strength fiber material is selected from the group consisting of: rayon, nylon, glass or Kevlar® (aramid) fiber.

5. The oxygen belt breathing pack, as described in claim 1, wherein said high strength ballistic ribbon material is selected from the group consisting of:
prepreg carbon fiber or prepreg glass fiber.

6. The oxygen belt breathing pack, as described in claim 1, wherein, said high pressure regulator is an integral portion of said manifold.

7. The oxygen belt breathing pack, as described in claim 1, wherein said manifold further includes an overpressure rupture fitting.

8. The oxygen belt breathing pack, as described in claim 1, wherein said at least one pressure vessel includes an overpressure rupture fitting.

9. The oxygen belt breathing pack, as described in claim 1, further comprising a pressure transducer, said pressure transducer detecting pressure levels within said at least one pressure vessel and comprising a wireless signal transmission mechanism.

10. The oxygen belt breathing pack, as described in claim 9, further comprising a remotely mounted pressure display device, said display device receiving wireless signals from said pressure transducer and displaying a pressure reading.

11. The oxygen belt breathing pack, as described in claim 1, wherein said at least one polymeric pressure vessel is formed as a seamless cylindrical body having semi-spherical ends, each of said ends having a central opening connected to said sections of flexible conduit.

12. The oxygen belt breathing pack, as described in claim 1, wherein said at least one polymeric pressure vessel is formed as a pair of two part shells, said shells being welded together and reinforced with a two part ring assembly.

13. The oxygen belt breathing pack, as described in claim 1, wherein said at least one polymeric pressure vessel is formed as an elongated tube with reduced diameter ends for connection to either of coupling, inlet and sealing fittings.

14. The oxygen belt breathing pack, as described in claim 13, wherein said at least one polymeric pressure vessel further comprising at least one intermediate section, said intermediate section having a reduced diameter to provide increased flexibility when forming said pressure vessel into a compact shape to fit a container.

15. The oxygen belt breathing pack, as described in claim 1, wherein said at least one polymeric pressure vessel is formed as an elongated, corrugated tube with reduced diameter ends for connection to either of coupling, inlet and sealing fittings.

16. The oxygen belt breathing pack, as described in claim 15, wherein said at least one polymeric pressure vessel further comprising at least one intermediate section, said intermediate section having a reduced diameter to provide increased flexibility when forming said pressure vessel into a compact shape to fit a container.

17. The oxygen belt breathing pack, as described in claim 1, further comprising a concentrator, said concentrator comprising:
a compressor, said compressor having a rotational power source, a low pressure input, a high pressure output;
at least one concentrator polymeric pressure vessel, said pressure vessel being fluidly connected to said high pressure output;
a high pressure regulator, said regulator being fluidly connected to said pressure vessel; and
a high pressure connection, said high pressure connection being fluidly connected to said high pressure regulator and connectable to an external pressure vessel.

18. The oxygen belt breathing pack, as described in claim 17, wherein said concentrator further comprises:
an inlet oxygen filter;
a pressure gauge, said pressure gauge measuring pressure in said at least one concentrator pressure vessel; and
a shut-off valve, said shut-off valve controlling said high pressure connection.

19. The oxygen belt breathing pack, as described in claim 17, wherein said at least one concentrator pressure vessel is formed as a seamless cylindrical body having semi-spherical ends, each of said ends having a central opening connected to said sections of flexible conduit.

20. The oxygen belt breathing pack, as described in claim 17, wherein said at least one concentrator pressure vessel is formed as a pair of two part shells, said shells being welded together and reinforced with a two part ring assembly.

21. The oxygen belt breathing pack, as described in claim 17, wherein said at least one concentrator pressure vessel is formed as an elongated, tube with reduced diameter ends for connection to either of coupling, inlet and sealing fittings.

22. The oxygen belt breathing pack, as described in claim 21, wherein said at least one concentrator pressure vessel further comprising at least one intermediate section, said intermediate section having a reduced diameter to provide increased flexibility when forming said pressure vessel into a compact shape to fit a container.

23. The oxygen belt breathing pack, as described in claim 17, wherein said at least one concentrator pressure vessel is formed as an elongated, corrugated tube with reduced diameter ends for connection to either of coupling, inlet and sealing fittings.

24. The oxygen belt breathing pack, as described in claim 23, wherein said at least one concentrator pressure vessel further comprising at least one intermediate section, said intermediate section having a reduced diameter to provide increased flexibility when forming said pressure vessel into a compact shape to fit a container.

25. The oxygen belt breathing pack, as described in claim 1, wherein said flexible container is in the form of a belt that can be worn about the waist of an ambulatory care patient.

26. The oxygen belt breathing pack, as described in claim 1, wherein said high pressure regulator includes a demand valve to supply oxygen directly to said nasal cannula at a reduced pressure.

27. The oxygen belt breathing pack, as described in claim 1, further comprising a pressure gauge, said gauge mounted to said manifold.
